(19) United States
(12) Patent Application Publication
(10) Pub. No.: US 2018/0169117 A1
(43) Pub. Date: Jun. 21, 2018

(54) PHARMACEUTICAL FORMULATION COMPRISING A SUBSTITUTED PHENYL - (1,3-DIHYDRO-ISOINDOL-2-YL) - METHANONE

(71) Applicant: OTSUKA PHARMACEUTICAL CO., LTD., Tokyo (JP)

(72) Inventors: David Alexander RAWLINS, Welwyn, Hertfordshire (GB); Sanjeev REDKAR, San Ramon, CA (US); Rajashree JOSHI-HANGAL, Pleasanton, CA (US)

(73) Assignee: OTSUKA PHARMACEUTICAL CO., LTD., Tokyo (JP)

(21) Appl. No.: 15/127,320

(22) PCT Filed: Mar. 19, 2015
(86) PCT No.: PCT/IB2015/000364

§ 371 Date: Sep. 19, 2016

Related U.S. Application Data
(60) Provisional application No. 61/955,979, filed on Mar. 20, 2014.

(51) Int. Cl. A61K 31/609; A61K 9/19; A61K 45/06; A61K 47/02; A61K 47/40; A61P 35/00

(52) U.S. Cl. CPC A61K 31/609; A61K 9/19; A61P 35/00; A61K 47/02; A61K 47/40; A61K 45/06

(57) ABSTRACT

Provided herein are formulations of (2,4-dihydroxy-5-isopropyl-phenyl)-[5-(4-methyl-piperazin-1-ylmethyl)-1,3-dihydro-isoindol-2-yl]-methanone of formula (I):

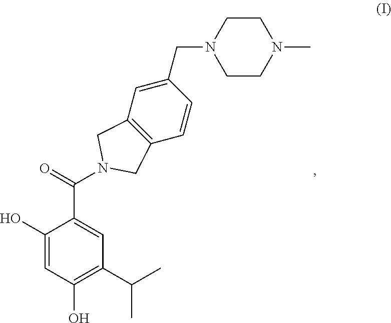 [SMILES: CC(C)c1cc(C(=O)N2Cc3ccc(CN4CCN(C)CC4)cc3C2)c(O)cc1O]

or a L-lactate salt thereof, in a phosphate or succinate buffer.

### FIELD OF THE INVENTION

The invention is directed to formulations of (2,4-dihydroxy-5-isopropyl-phenyl)-[5-(4-methyl-piperazin-1-ylmethyl)-1,3-dihydro-isoindol-2-yl]-methanone, or a salt thereof, with a phosphate or succinate buffer and having improved solubility.

### BACKGROUND OF THE INVENTION

(2,4-Dihydroxy-5-isopropyl-phenyl)-[5-(4-methyl-piperazin-1-ylmethyl)-1,3-dihydro-isoindol-2-yl]-methanone (hereinafter referred to as the compound of formula (I)) and its salts are disclosed in U.S. Pat. No. 7,700,625 as being inhibitors of the heat shock protein Hsp90. These compounds are useful for the treatment of Hsp90-mediated diseases such as cancer.

Current formulations of the lactic acid salt of the compound of formula (I) include unbuffered solutions at pH 4.0. It has been observed, however, that when pH increases, solubility of the lactic acid salt of the compound of formula I decreases. Thus, there is a need for additional formulations of the compound of formula I, or its salts, having improved solubility at relatively higher pH values for intravenous administration.

### SUMMARY OF THE INVENTION

The present invention is directed to a pharmaceutical formulation, comprising (2,4-dihydroxy-5-isopropyl-phenyl)-[5-(4-methyl-piperazin-1-ylmethyl)-1,3-dihydro-isoindol-2-yl]-methanone of formula (I):

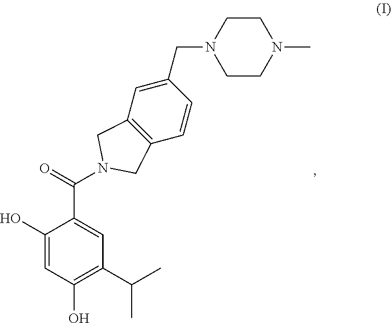 [SMILES: CC(C)c1cc(C(=O)N2Cc3ccc(CN4CCN(C)CC4)cc3C2)c(O)cc1O]

or a L-lactate salt thereof, and a phosphate or succinate buffer.

The present invention is also directed to a method of treating cancer, comprising the step of administering a therapeutically effective amount of the above pharmaceutical formulation to a patient in need thereof.

### DETAILED DESCRIPTION OF THE INVENTION

The compound of formula (I) is disclosed in U.S. Pat. No. 7,700,625. It has unexpectedly been discovered that the solubility of the compound of formula (I) or its salts increases at relatively high pH ranges when in the presence of phosphate buffer or succinate buffer. It has also surprisingly been discovered that not all buffers are useful to improve the solubility of this compound or its salts at relatively high pH ranges.

Thus, in one embodiment, the present invention provides a pharmaceutical formulation, comprising (2,4-dihydroxy-5-isopropyl-phenyl)-[5-(4-methyl-piperazin-1-ylmethyl)-1,3-dihydro-isoindol-2-yl]-methanone of formula (I):

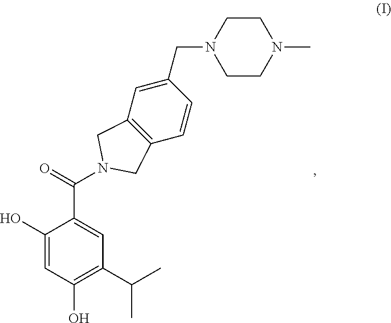 [SMILES: CC(C)c1cc(C(=O)N2Cc3ccc(CN4CCN(C)CC4)cc3C2)c(O)cc1O]

or a L-lactate salt thereof, and a phosphate or succinate buffer.

The formulations of the invention may be referred to for convenience herein as “the formulations of the invention” or “the pharmaceutical formulations of the invention” or “the buffered formulations of the invention”.

The formulations of the invention can be provided in liquid form so that they can, for example, be administered parenterally. Thus, for example, the formulations can be formulated as solutions or suspensions which are suitable for administration by injection or infusion.

In one embodiment, the formulations of the invention are solutions.

Alternatively, the formulations of the invention can be provided in a dry form that can be mixed with a suitable liquid carrier (e.g. an aqueous carrier such as sterile water or water for injection) to give a formulation in liquid form as defined above. For example, the formulations of the invention can be provided in the form of a powder, or granules or in lyophilized form.

In one particular embodiment, the formulations of the invention are provided in lyophilized form.

The liquid formulations of the invention can contain an amount of L-lactate salt of the compound of formula (I) from about 0.5 mg/ml to about 120 mg/ml; for example from about 1.0 mg/mL to about 100 mg/ml, or about 10 mg/ml to about 96 mg/ml, or about 20 mg/ml to about 80 mg/ml; or about 40 mg/ml to about 60 mg/ml; or about 45 mg/ml to about 55 mg/ml; for example approximately 50 mg/ml.

When the formulations of the invention are in dry form, they may contain an amount of the L-lactate salt of the compound of formula (I) which is sufficient to provide a concentration in the range from about from about 0.5 mg/ml to about 120 mg/ml (for example about 1.0 mg/mL to about 95.7 mg/mL), or from about 1.0 mg/mL to about 100 mg/ml, or about 10 mg/ml to about 96 mg/ml, or about 20 mg/ml to about 80 mg/ml; or about 40 mg/ml to about 60 mg/ml; or about 45 mg/ml to about 55 mg/ml; for example approximately 50 mg/ml of the compound of formula (I) when the formulation is mixed with a liquid carrier such as an aqueous carrier, e.g. 0.9% saline, 5% dextrose or water for injection.

In one embodiment, the buffer is a phosphate buffer.

In another embodiment, the buffer is a succinate buffer.

In a further embodiment, the buffer is a mixed phosphate/succinate buffer.

The phosphate buffers may optionally contain other buffering agents in addition to phosphate. For example, the phosphate buffers may contain borate or citrate. In one embodiment, however, the phosphate buffers contain no other buffering agents.

The phosphate buffer used in the formulations of the present invention may be an alkaline metal or alkaline earth metal phosphate buffer such as a sodium phosphate buffer.

Accordingly, in another embodiment, the present invention provides a pharmaceutical formulation of the invention, wherein the phosphate buffer is a sodium phosphate buffer.

The sodium phosphate used to prepare the formulations of the invention can be, for example, sodium dihydrogen phosphate or disodium hydrogen phosphate or mixtures thereof.

The sodium phosphate (e.g. the sodium dihydrogen phosphate or disodium hydrogen phosphate or mixtures thereof) can be used in anhydrous form, or in hydrated forms, or mixtures of anhydrous and hydrated forms. For example, sodium dihydrogen phosphate may be used in the form of its monohydrate whereas di-sodium hydrogen phosphate may be used in the form of its dihydrate.

Therefore, in one embodiment, the phosphate buffer is sodium dihydrogen phosphate (e.g. the monohydrate thereof).

In another embodiment, the phosphate buffer is di-sodium hydrogen phosphate (e.g. the dihydrate thereof).

In a further embodiment, the phosphate buffer is a combination of more than one sodium phosphate buffer. For example, the phosphate buffer can be a combination of two sodium phosphate buffers. In one particular embodiment, the formulation contains first and second sodium phosphate buffers wherein the first sodium phosphate buffer is sodium dihydrogen phosphate (e.g. in monohydrate form) and the second sodium phosphate buffer is di-sodium hydrogen phosphate (e.g. in dihydrate form).

It will be appreciated that the proportions of sodium dihydrogen phosphate and disodium hydrogen phosphate can be varied to provide a desired pH value for the formulation. Where necessary, acid or base may be added to make adjustments to the final pH.

Similarly, where only one of sodium dihydrogen phosphate and disodium hydrogen phosphate is used to prepare the formulation, acid or base may be added to adjust the pH of the formulation to the required value.

Succinate buffers can be prepared by dissolving succinic acid in water and then adding a base (e.g. an alkali metal or alkaline earth metal hydroxide such as sodium hydroxide) to give a desired pH.

In another embodiment, the pharmaceutical formulation of the invention is one which has had an acid or a base added thereto. The acid or the base is used to adjust the pH value of the pharmaceutical composition. The formulations will therefore contain anions or cations characteristic of the acids or bases that have been added to the formulation.

Accordingly, in one embodiment, there is provided a pharmaceutical formulation of the invention, wherein the added acid is hydrochloric acid, and therefore chloride ions are present in the formulation.

In another embodiment, there is provided a pharmaceutical formulation of the invention wherein the added base is sodium hydroxide, and therefore sodium ions are present in the formulation.

The formulations of the invention are typically formulated so that, when presented in liquid form or when added to a liquid carrier to give a liquid form, they have a pH in the range from about 4.6 to about 5.4, for example from about 4.8 to 5.4, or about 4.8 to about 5.2.

Accordingly, in another embodiment, the pharmaceutical formulation of the invention is at a pH of about 4.6 to about 5.4.

In another embodiment, the pharmaceutical formulation of the invention is at a pH of about 4.8 to about 5.2.

In another embodiment, the pharmaceutical formulation of the invention is at a pH of about 4.8 to about 5.4.

The concentrations of phosphate buffer or succinate buffer needed to provide a formulation of the desired pH will typically range from about 50 mM to about 250 mM.

Therefore, in another embodiment of the pharmaceutical formulations of the invention, the phosphate buffer or succinate buffer is at a concentration of about 50 mM to about 250 mM.

In another embodiment, the phosphate buffer or succinate buffer is at a concentration of about 50 mM.

In another embodiment, the phosphate buffer or succinate buffer is at a concentration of about 100 mM.

In another embodiment, the phosphate buffer or succinate buffer is at a concentration of about 200 mM.

The pharmaceutical formulations of the invention may initially be prepared as bulk liquid solutions and then lyophilised to give a dry powder formulation that can subsequently be reconstituted by mixing with a carrier liquid (e.g. an aqueous carrier liquid) prior to administration.

Bulk liquid formulations that can be lyophilised to a dry powder formulation represent a further embodiment of the invention.

In a particular embodiment, the present invention provides a pharmaceutical liquid formulation suitable for lyophilization to give a reconstitutable powder, wherein the formulation comprises an aqueous solution containing:

about 48 mg/mL to about 52 mg/mL (e.g. about 50 mg/mL) (free base equivalent) of the L-lactate salt of (2,4-dihydroxy-5-isopropyl-phenyl)-[5-(4-methyl-piperazin-1-ylmethyl)-1,3-dihydro-isoindol-2-yl]-methanone;

about 24 mg/mL to about 27 mg/mL (e.g. about 25.59 mg/mL) of sodium dihydrogen phosphate monohydrate (or an equivalent amount of the anhydrous or dihydrate forms); and

about 1.75 mg/mL to about 2.75 mg/mL (e.g. about 2.28 mg/mL) of di-sodium hydrogen phosphate dihydrate (or an equivalent amount of the anhydrous or monohydrate forms).

The bulk solution is subjected to lyophilisation. Thus, containers (e.g. vials) are filled with an amount (e.g. approximately 5.3 mL) of the bulk solution and then freeze dried to produce the drug product. Vials of drug product are reconstituted, for example with 10 mL of water for injection, 0.9% sodium chloride or 5% dextrose solutions, to give concentrations of the individual components that are approximately half those in the bulk solution.

Lyophilised formulations constitute a further embodiment of the invention.

Accordingly, in another embodiment of the present invention, there is provided a pharmaceutical formulation in dry lyophilised form comprising (2,4-dihydroxy-5-isopropyl-phenyl)-[5-(4-methyl-piperazin-1-ylmethyl)-1,3-dihydro-isoindol-2-yl]-methanone L-lactate salt and a phosphate buffer (e.g. a sodium phosphate buffer as defined herein), the phosphate buffer being present in an amount such that when the formulation is reconstituted in an aqueous liquid carrier for injection or infusion to give a solution containing a concentration of 20 mg/ml to 30 mg/ml (e.g. approximately 25 mg/ml) of (2,4-dihydroxy-5-isopropyl-phenyl)-[5-(4-methyl-piperazin-1-ylmethyl)-1,3-dihydro-isoindol-2-yl]-methanone L-lactate salt, the solution has a pH in the range from about 4.6 to about 5.4.

In another embodiment of the present invention, there is provided a method of preparing a pharmaceutical formulation in lyophilised form, which method comprises forming a solution of (2,4-dihydroxy-5-isopropyl-phenyl)-[5-(4-methyl-piperazin-1-ylmethyl)-1,3-dihydro-isoindol-2-yl]-methanone L-lactate salt in an aqueous carrier containing a phosphate buffer as defined herein, wherein in the solution has a pH in the range from about 4.8 to about 5.2, and then lyophilising the solution.

The method may comprise one or more pH checking and/or adjustment steps. Adjustment of the pH may be accomplished by adding an acid such as hydrochloric acid or a base such as sodium hydroxide. For example, the phosphate buffer may be dissolved in the aqueous carrier to give a buffered solution, the pH of the buffered solution measured and, where necessary adjusted to a desired pH, and the (2,4-dihydroxy-5-isopropyl-phenyl)-[5-(4-methyl-piperazin-1-ylmethyl)-1,3-dihydro-isoindol-2-yl]-methanone L-lactate salt added to the buffered solution. Thereafter, the pH of the solution may be measured and, if necessary, adjusted.

Thus, prior to lyophilisation, an acid or base (e.g. hydrochloric acid or sodium hydroxide) may be added to the bulk formulation to bring the pH to the desired value. For example, an amount of 1M hydrochloric acid (or a higher volume of a less concentrated solution of HCl such as 0.5M) may be added to bring the pH of the formulation to about 5.0. Alternatively, an amount of 1M sodium hydroxide (or a higher volume of a less concentrated solution of NaOH such as 0.5M) may be added to bring the pH of the formulation to about 5.0.

The method may comprise one or more filtration steps prior to lyophilisation of the solution. For example, the solution may be sterile filtered and then filled into one or more containers (e.g. vials) for lyophilisation.

Therapeutic Uses

The pharmaceutical formulations as defined herein can be administered as the sole therapeutic agent or they can be administered in combination therapy with one of more other compounds (also referred to herein as “ancillary compounds”) for treatment of a particular disease state, for example a neoplastic disease such as a cancer as defined herein. Examples of other therapeutic agents or treatments that may be administered together (whether concurrently or at different time intervals) with the compounds of the formula (I) include, but are not limited to: Topoisomerase I inhibitors, antimetabolites, tubulin targeting agents, DNA binder and topoisomerase II inhibitors, alkylating agents, monoclonal antibodies, anti-hormones, signal transduction inhibitors, proteasome inhibitors, DNA methyl transferases, cytokines and retinoids, chromatin targeted therapies, e.g. HDAC or HAT modulators, and radiotherapy.

In a further embodiment, the present invention provides a method for treating cancer, comprising the step of administering a therapeutically effective amount of a pharmaceutical formulation comprising (2,4-dihydroxy-5-isopropyl-phenyl)-[5-(4-methyl-piperazin-1-ylmethyl)-1,3-dihydro-isoindol-2-yl]-methanone of formula (I):

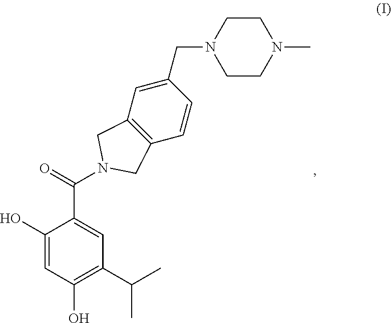 [SMILES: CC(C)c1cc(C(=O)N2Cc3ccc(CN4CCN(C)CC4)cc3C2)c(O)cc1O]

or a L-lactate salt thereof, and a phosphate or succinate buffer to a patient in need thereof.

In a further embodiment, the present invention provides a pharmaceutical formulation of the invention for use in treating cancer, the formulation comprising a therapeutically effective amount of (2,4-dihydroxy-5-isopropyl-phenyl)-[5-(4-methyl-piperazin-1-ylmethyl)-1,3-dihydro-isoindol-2-yl]-methanone or a L-lactate salt thereof, and a phosphate or succinate buffer as defined herein.

In a further embodiment, the present invention provides a method for treating cancer (or a pharmaceutical formulation of the invention for use in treating cancer), wherein the cancer is selected from head and neck cancer, carcinoma of the bladder, breast, colon, kidney, epidermis, liver, lung, ovary, pancreas, stomach, thyroid, prostate, gastrointestinal system, skin, a hematopoietic tumor of lymphoid or myeloid lineage, and a tumor of the central or peripheral nervous system.

In a further embodiment, the present invention provides a method for treating cancer (or a pharmaceutical formulation of the invention for use in treating cancer), wherein the cancer is selected from: colon adenocarcinoma, colon adenoma, colorectal carcinoma, small cell lung cancer, non-small cell lung carcinoma, exocrine pancreatic carcinoma, gastrointestinal stromal tumors, leukemia, acute lymphocytic leukemia, chronic lymphocytic leukemia, B-cell lymphoma, T-cell lymphoma, Burkett's lymphoma, acute myelogenous leukemia, chronic myelogenous leukemia, Imatinib sensitive and refractory chronic myelogenous leukemia, myeloproliferative disease, melanoma, bortezomib sensitive multiple myeloma, thyroid follicular cancer and glioma.

In a further embodiment, the present invention provides a method for treating cancer (or a pharmaceutical formulation of the invention for use in treating cancer), wherein the cancer is selected from: carcinoma of the prostate, gastrointestinal stromal tumors, acute lymphocytic leukemia, chronic lymphocytic leukemia, B-cell lymphoma, T-cell lymphoma, Burkett's lymphoma, acute myelogenous leukemia, chronic myelogenous leukemia, bortezomib sensitive multiple myeloma, non-small cell lung cancer, thyroid cancer, follicular cancer, melanoma, and ErbB2-positive breast cancer.

In a further embodiment, there is provided a method for treating cancer (or a pharmaceutical formulation of the invention for use in treating cancer) wherein the cancer is selected from metastatic breast cancer which is HER2 positive; adenocarcinoma of the prostate; metastatic melanoma; non-small cell carcinoma of the lung (NSCLC); small cell carcinoma of the lung (SCLC); high grade gliomas; gastrointestinal stromal tumors (GIST); colorectal cancer; glioblastoma; melanoma; metastatic thyroid cancer; prostate cancer; and rectal cancer.

Within this group of cancers, a particular subgroup consists of colorectal cancer; glioblastoma; melanoma; metastatic thyroid cancer; prostate cancer; and rectal cancer.

In a further embodiment of the invention, there is provided a method for treating cancer (or a pharmaceutical formulation of the invention for use in treating cancer) wherein the cancer is selected from ErbB2-positive breast, prostate, lung, and gastric cancer; chronic myeloid leukemia; androgen receptor dependent prostate cancer; Flt3-dependent acute myeloid leukaemia; melanoma associated with BRAF mutation; multiple myeloma; velcade refractory multiple myeloma; and gastrointestinal stromal tumours (GIST). Within this group of cancers, a particular subset consists of multiple myelomas and velcade refractory tumour types.

In a further embodiment of the invention, there is provided a method for treating cancer (or a pharmaceutical formulation of the invention for use in treating cancer) wherein the cancer is selected from hormone refractory prostate cancer, metastatic melanoma, HER2 positive breast cancer, mutant EGFR positive non-small cell lung carcinoma, Small Cell Lung Carcinoma and Gleevec resistant gastrointestinal stromal tumours.

In a further embodiment of the invention, there is provided a method for treating cancer (or a pharmaceutical formulation of the invention for use in treating cancer) wherein the cancer is selected from refractory solid tumours, gastrointestinal stromal tumours (GIST), prostate cancer, melanoma (e.g. melanoma associated with BRAF mutation), non-small cell lung cancer (e.g. ALK-positive non-small cell lung cancer), HER2-positive breast cancer; and multiple myeloma. Within this group of cancers, a particular subset consists of refractory solid tumours, gastrointestinal stromal tumours (GIST), prostate cancer, melanoma associated with BRAF mutation, and ALK-positive non-small cell lung cancer.

Based on the activities of Hsp90 client proteins and experimental evidence, the following disorders may be particularly sensitive to treatment with the pharmaceutical formulations of the present invention:

ErbB2-Positive Breast, Prostate, Lung, and Gastric Cancer

Overexpression of ErbB2 (HER-2) occurs in approximately 30% of breast cancers and is linked to poor prognosis and drug resistance (Tsugawa et. al., 1993. Oncology 1993; 50: 418).

Mutant EGFR in Lung Cancer

Somatic mutations in the kinase domain of the epidermal growth factor receptor (EGFR), including L858R and exon 19 deletions, underlie responsiveness to gefitinib and erlotinib in non-small cell lung cancer (NSCLC). Acquired resistance to these tyrosine kinase inhibitors is in some cases mediated by a second mutation, T790M. Ansamycin antibiotics, such as geldanamycin, potently inhibit heat shock protein 90 (Hsp90), promoting ubiquitin-mediated degradation of oncogenic kinases that require the chaperone for proper conformational folding. Exposure of EGFR-mutant cell lines to geldanamycin induced marked depletion of phospho-Akt and cyclin D1 as well as apoptosis. These data suggest mutational activation of EGFR is associated with dependence on Hsp90 for stability and that Hsp90 inhibition may represent a novel strategy for the treatment of EGFR-mutant NSCLC.

Chronic Myeloid Leukemia

The aberrant BCR-Abl protein is created through a chromosomal translocation and results in a constitutively active Abl kinase domain. This translocation event has been shown to be causal for CML. P210BcrAbl is a known client protein for Hsp90. Treatment of the BCR-Abl positive cell line K562 with an hsp90 inhibitor induced apoptosis. The Bcr-Abl inhibitor Gleevec® also induces apoptosis in K562 cells; however Gleevec® resistant K562 cells still retain sensitivity towards Hsp90 inhibitors (Gone et. al. 2002, Blood 100: 3041-3044).

Androgen Receptor Dependent Prostate Cancer

The androgen receptor kinase is an Hsp90 client protein. Testosterone remains the primary therapy for non-localised disease although the development of resistance is inevitable. In some cases resistance develops as a consequence of a mutation occurring in the androgen receptor conferring ligand-independent signaling. Under these circumstances down regulation of androgen receptor expression following Hsp90 inhibition represents a potential therapeutic approach. A parallel system exists in estrogen-dependent breast cancers.

Flt3-Dependent Acute Myeloid Leukaemia

Internal duplication of the tyrosine kinase receptor Flt3 leads to its constitutive activation and oncogenesis. These internal duplications are observed in 20% of all reported cases of AML and are an indication of poor prognosis. Inhibition of Flt3 signaling has been shown to lead to transient responses. Hsp90 inhibitors are predicted to be of clinical benefit to these patients as Flt3 is an Hsp90 client protein (Bali et. al., 2004 Cancer Res. 64(10):3645-52).

Melanoma Associated with BRAF Mutation

BRAF encodes for a serine/threonine kinase which is mutated in 70% of all melanomas. 80% of these represent a single V599E point mutation that confers elevated kinase activity to BRAF. This mutation is also transforming in NIH3T3 cells (Bignell et. al., 2002 Nature. 417(6892):949-54).

Multiple Myeloma

The Hsp90 inhibitor 17-AAG potently inhibits proliferation of Bortezomib refractory multiple myeloma cell lines. Cell surface levels of IGF-1R and IL-6R were also diminished in 17-AAG treated MM-1 cells (Mitsiades et. al., Blood 107:1092-1100, 2006). Autocrine stimulation of multiple myeloma cells, as well as paracrine stimulation of bone marrow stromal cells with IL-6 is also diminished through downregulation of the Hsp90 client IKK.

Bortezomib (Velcade) Refractory Cancers

Compounds of the present invention may be used in the treatment of Velcade refractory tumour types including treatment of patients with multiple myeloma, mantle cell lymphoma, indolent non-Hodgkin's lymphoma, stage IIIB and IV Bronchioloalveolar carcinoma, advanced non-small cell lung cancer, breast, prostate and ovarian cancers and non-Hodgkin's lymphoma.

Gastrointestinal Stromal Tumours (GIST)

GIST tumours are particularly disease dependent on growth factor activation or overexpression (e.g. c-kit).

In a further embodiment of the present invention, there is provided the use of a pharmaceutical formulation comprising a therapeutically effective amount of (2,4-dihydroxy-5-isopropyl-phenyl)-[5-(4-methyl-piperazin-1-ylmethyl)-1,3-dihydro-isoindol-2-yl]-methanone or a L-lactate salt thereof, and a phosphate buffer as defined herein for the manufacture of a medicament for the treatment of cancer, for example any cancer or group or subset of cancers as defined above and elsewhere herein.

In other embodiments, the present invention provides the use of a combination of (2,4-dihydroxy-5-isopropyl-phenyl)-[5-(4-methyl-piperazin-1-ylmethyl)-1,3-dihydro-isoindol-2-yl]-methanone or a L-lactate salt thereof, and a phosphate buffer for the manufacture of a medicament for use in each of the foregoing methods of treatment.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “pharmaceutical formulation” (or “formulation”) means a mixture or solution containing a therapeutically effective amount of at least one active pharmaceutical ingredient (“API”) together with pharmaceutically acceptable excipients to be administered to a mammal, e.g., a human in need thereof.

The term “pharmaceutically acceptable excipient” is used herein in its conventional sense and refers to an ingredient which typically has no significant therapeutic activity and has acceptable toxicity such as buffers, solvents, tonicity agents, stabilizers, antioxidants, surfactants or polymers used in formulating pharmaceutical products. They are generally safe for administering to humans according to established governmental standards, including those promulgated by the United States Food and Drug Administration.

The term “buffer” as used herein denotes a pharmaceutically acceptable excipient, which stabilizes the pH of a pharmaceutical preparation. A particular pharmaceutically acceptable buffer is a phosphate buffer. Examples of phosphate buffers include magnesium phosphate buffers, potassium phosphate buffers and sodium phosphate buffers. A particular phosphate buffer is a sodium phosphate buffer. Examples of sodium phosphate buffers include sodium dihydrogen phosphate (e.g. the monohydrate) and di-sodium hydrogen phosphate (e.g. the dehydrate), or combinations thereof. The concentration of phosphate buffers used may be from about 50 mg/mM to about 250 mg/mM. The pH of the formulation can be adjusted before or after the API is added.

The pharmaceutical formulations of the present invention can optionally include an acid and/or a base to adjust the pH of the formulation to a desired value. Thus, independently from the buffer used, the pH can be adjusted with an acid or a base known in the art, e.g. hydrochloric acid, acetic acid, phosphoric acid, sulfuric acid and citric acid, sodium hydroxide and potassium hydroxide. An example of an acid is 1.0 M HCl and an example of a base is 1.0M NaOH.

The formulations may be presented in unit-dose or multi-dose containers, for example sealed ampoules and vials, and may be stored in a freeze-dried (lyophilised) condition requiring only the addition of the sterile liquid carrier for reconstitution, for example water for injections, immediately prior to use. In an embodiment of the invention, the contents of one or more (e.g. two) vials at appropriate dosages can be administered. For example, in a once weekly regime, two vials are typically administered.

The pharmaceutical formulation can be prepared by lyophilising a compound of Formula (I) or a L-lactate salt thereof. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, for example less than 5% by weight, e.g. less than 1% by weight, based on the weight of the lyophile.

The pH of the lyophilized composition is typically from about 4.6 to about 5.4. Upon reconstitution with water for injection, 0.9% saline or 5% dextrose, the pH is typically from about 4.8 to about 5.2.

The formulations of the invention can optionally include one or more auxiliary pharmaceutically acceptable excipients such as surfactants, emulsifiers and cyclodextrins.

Examples of surfactants are physiologically acceptable non-ionic surfactants such as polyoxyethylene sorbitan esters, for example polyoxyethylene sorbitan monooleate (polysorbate 80 or “Tween”). In an embodiment of the invention, the amount of polysorbate 80 (Tween-80) in the pharmaceutical formulation can be from 1.0 to 8.0%. It has been found that, at physiological pH, addition of 1-8 (w/v) Tween resulted in an approximately 1.5 fold (50%) increase in solubility of the lactate salt of the compound of formula (I).

The lyophilisation formulation may contain other excipients for example, thickening agents, dispersing agents, antioxidants, preservatives, and tonicity adjusters. Examples of antioxidants include ascorbic acid, sodium bisulphite, sodium metabisulphite, monothioglycerol, thiourea, butylated hydroxytoluene, butylated hydroxyl anisole, and ethylenediaminetetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride.

Bulking agents are generally used in lyophilisation technology for facilitating the process and/or providing bulk and/or mechanical integrity to the lyophilized cake. A bulking agent is a freely water soluble, solid particulate diluent which, when co-lyophilised with the compound or salt thereof, provides a physically stable lyophilized cake, a more optimal freeze-drying process and rapid and complete reconstitution. The bulking agent may also be used to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively, the formulations of the invention can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery; for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

The formulation will generally be presented in unit dosage form (e.g. in a vial) and, as such, will typically contain sufficient compound to provide a desired level of biological activity. For example, a unit dosage form of a formulation of the invention may contain from 0.1 milligrams to 2 grams of active ingredient, e.g. from 1 milligram to 1.5 gram of active ingredient. Within this range, particular sub-ranges of compound are 10 milligrams to 1 gram of active ingredient (more usually from 20 to 800 milligrams, e.g. 50 to 500 milligrams, or 100 to 400 milligrams, or 200 to 300 milligrams, or 240 to 300 milligrams, for example approximately 265 milligrams, of active ingredient).

Methods of Treatment

It is envisaged that the formulations of the invention as defined herein will be useful in the prophylaxis or treatment of a range of disease states or conditions mediated by Hsp90 client proteins. Examples of such disease states and conditions are set out above.

The formulations are generally administered to a subject in need of such administration, for example a human or animal patient, preferably a human.

The formulations will typically be administered in amounts that are therapeutically or prophylactically useful and which generally are non-toxic. However, in certain situations (for example in the case of life threatening diseases), the benefits of administering the formulation may outweigh the disadvantages of any toxic effects or side effects, in which case it may be considered desirable to administer formulations in amounts that are associated with a degree of toxicity.

The formulations may be administered over a prolonged term to maintain beneficial therapeutic effects or may be administered for a short period only. Alternatively they may be administered in a pulsatile or continuous manner.

The formulations can be administered so as to provide a typical daily dose of the compound of formula (I) in the range from 100 (3500) picograms to 100 (3500) milligrams per kilogram of body weight, more typically 5 (175) nanograms to 25 (875) milligrams per kilogram of bodyweight, and more usually 10 (350) nanograms to 15 (525) milligrams per kilogram (e.g. 10 (350) nanograms to 10 (350) milligrams, and more typically 1 (35) microgram per kilogram to 20 (700) milligrams per kilogram, for example 1 (35) microgram to 10 (350) milligrams per kilogram) per kilogram of bodyweight although higher or lower doses may be administered where required (the figures shown in parenthesis denote equivalent doses expressed on a mg/m² basis based on a 70 kg person with a Body Surface Area of 2.0 m²). The compound can be administered on a daily basis or on a repeat basis every 2, or 3, or 4, or 5, or 6, or 7, or 10 or 14, or 21, or 28 days for example.

In one particular dosing schedule, a patient will be given an infusion of a formulation of the invention for periods of one hour daily for up to ten days in particular up to five days for one week, and the treatment repeated at a desired interval such as two to four weeks, in particular every three weeks.

More particularly, a patient may be given an infusion of a formulation of the invention for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

In a still further dosing schedule, a patient is given a formulation which provides 220-260 mg/m² (e.g. approximately 260 mg/m²) of active ingredient over a one hour period, once per week for three weeks out of four.

Ultimately, however, the dosing schedule used will be commensurate with the nature of the disease or physiological condition being treated and will be at the discretion of the physician.

Combination Therapy

In a particular embodiment, the pharmaceutical formulations of the present invention can also include one or more ancillary compounds as disclosed and defined in U.S. Pat. No. 8,277,807. Examples of such ancillary compounds include:

I. corticosteroids, antiandrogens, antiestrogens, aromatase inhibitors and GNRAs;

II. interferons and interleukins;

III. tretinoin, alitretinoin and bexarotene;

IV. rituximab, tositumomab and gemtuzumab ozogamicin; alemtuzumab, alemtuzumab, and bevacizumab;

V. camptothecin compounds;

VI. 5-fluorouracil, capecitabine, gemcitabine, cytarabine, fludarabine, raltitrexed, pemetrexed and methotrexate;

VII. vinca alkaloids;

VIII. taxanes;

IX. epothilones;

X. platinum compounds;

XI. anthracycline derivatives, mitoxantrone and podophyllotoxin derivatives;

XII. aziridines, nitrogen mustards, nitrosurea alkylating agents and bisalkanesulfonates;

XIII. CDK inhibitors;

XIV. COX-2 inhibitors;

XV. trichostatin A, suberoylanilide hydroxamic acid, JNJ-16241199, LAQ-824, MGCD-0103, PXD-101; XVI. selective immunoresponse modulators;

XVII. temozolomide, decitabine, 5-azacitidine, pseudoisocytidine and 5-fluoro-2′-deoxycytidine;

XVIII. bortezimib and bleomycin;

XIX. aurora inhibitors;

XX. herbimycin, geldanamycin, 17-AAG, 17-DMAG, CNF-2024, and IPI-504;

XXI. checkpoint targeting agents;

XXII. DNA repair inhibitors;

XXIII inhibitors of G-protein coupled receptor inhibitors;

XXIV. trastuzumab, cetuximab, panitumumab, tipifamib, gefitinib, erlotinib, bevacizumab, sunitinib, imatinib mesylate, sorafenib dasatinib, lapatinib, nilotinib, vandetanib, vatalinib and CHIR-258 and

XXV. anti-emetic agents, agents that prevent or decrease the duration of chemotherapy associated neutropenia and prevent complications that arise from reduced levels of red blood cells or white blood cells, agents that inhibit bone resorption, bisphosphonate agents, agents that suppress inflammatory responses, agents that reduce blood levels of growth hormone and IGF-I in acromegaly patients, antidotes to drugs that decrease levels of folic acid, and agents for the treatment of oedema and thromboembolic episodes.

Examples of such ancillary compounds I include tamoxifen; toremifene; raloxifene; medroxyprogesterone; megestrol/megestrel; aminoglutethimide; letrozole; anastrozole; exemestane; goserelin; leuprolide; abarelix; fluoxymestrone; diethylstilbestrol; ketoconazole; fulvestrant; flutamide; bicalutimide; nilutamide; cyproterone and buserelin.

Examples of ancillary compounds II include interferon .alpha.-2b, interferon .alpha.-2a, Proleukin® IL-2, Picibanil, Romurtide, Sizofuran, Virulizin and Thymosin alpha 1.

Examples of ancillary compounds III include tretinoin, alitretinoin and bexarotene.

Examples of ancillary compounds IV include rituximab, tositumomab and gemtuzumab ozogamicin; alemtuzumab, alemtuzumab, and bevacizumab;

Examples of ancillary compounds V include irinotecan and topotecan.

Examples of ancillary compounds VI include 5-fluorouracil, capecitabine, gemcitabine, cytarabine, fludarabine, raltitrexed, pemetrexed and methotrexate;

Examples of ancillary compounds VII include vindesine, vinvesir, vinblastine, vincristine and vinorelbine.

Examples of ancillary compounds VIII paclitaxel and docetaxel.

Examples of ancillary compounds IX include ixabepilone, patupilone, BMS-247550 and desoxyeopthilone.

Examples of ancillary compounds X include cisplatin, carboplatin and oxaliplatinchloro(diethylenediamino)-platinum (II) chloride; dichloro(ethylenediamino)-platinum (II); spiroplatin; iproplatin; diamino(2-ethylmalonato)platinum (II); (1,2-diaminocyclohexane)malonatoplatinum (II); (4-carboxyphthalo)-(1,2-diaminocyclohexane)platinum (II); (1,2-diaminocyclohexane) (isocitrato)platinum (II); (1,2-diaminocyclohexane)-cis-(pyruvato)platinum (II); onnaplatin; and tetraplatin.

Examples of ancillary compounds XI include anthracycline derivatives, mitoxantrone and podophyllotoxin derivatives.

Examples of ancillary compounds XII include cyclophosphamide, ifosfamide/ifosphamide, chlorambucil, carmustine and lomustine, mitomycin, busulfan, estramustine, mechlorethamine, melphalan, bischloroethylnitrosurea, cyclohexylchloroethylnitrosurea, methylcyclohexylchloroethylnitrosurea, nimustine, procarbazine, dacarbazine, temozolimide and thiotepa.

Examples of ancillary compounds XIII include seliciclib, alvocidib, 7-hydroxy-staurosporine, PHA533533 and PD332991.

Examples of ancillary compounds XIV include celecoxib, arcoxia and lumiracoxib.

Examples of ancillary compounds XV include trichostatin A, suberoylanilide hydroxamic acid, PXD-101; XVI. lenalidomide and thalidomide.

Examples of ancillary compounds XVII include temozolomide, decitabine, 5-azacitidine, pseudoisocytidine and 5-fluoro-2′-deoxycytidine.

Examples of ancillary compounds XVIII include bortezimib and bleomycin.

Examples of ancillary compounds XIX include PHA-739358.

Examples of ancillary compounds XX include IPI-504.

Examples of ancillary compounds XXI include bendamustine, BSI-201 and AG-014699.

Examples of ancillary compounds XXII include atrasentan;

Examples of ancillary compounds XXIII include trastuzumab, cetuximab, panitumumab, tipifarnib, gefitinib, erlotinib, bevacizumab, sunitinib, imatinib mesylate, sorafenib dasatinib, lapatinib, nilotinib, vandetanib, vatalinib and CHIR-258.

Examples of ancillary compounds XXIV include erythropoietin, granulocyte macrophage-colony stimulating factor, granulocyte colony stimulating factor, zoledronate, pamidronate, ibandronate, dexamethazone, prednisone, prednisolone, octreotide acetate, leucovorin, folinic acid and megestrol acetate.

Combinations of pharmaceutical formulations of the present invention with platinum agents, taxol, taxotere, gemcitabine, pemetrexed, mitomycin, ifosfamide, vinorelbine, erlotinib and bevacizumab or pharmaceutical formulations of the present invention with carboplatin and taxol or cisplatin and gemcitabine are particularly suitable for treating Non-Small cell lung cancer.

Combinations of pharmaceutical formulations of the present invention with 5-FU, leucovorin and CPT 11 or a combination of a pharmaceutical formulation of the present invention with 5-FU, leucovorin and oxaliplatin, each with bevacizumab are particularly suitable for treating colon cancer.

Particularly suitable for treating breast cancer are combinations of pharmaceutical formulations of the present invention with (a) monoclonal antibodies (e.g. trastuzumab and bevicizamab); (b) monoclonal antibodies (e.g. trastuzumab and bevicizamab) and taxanes; and (c) antimetabolites (e.g. capecitabine) and signaling inhibitors (e.g. lapatinib).

Further combinations suitable for treating breast cancer are combinations of pharmaceutical formulations of the present invention with 5-FU, doxorubicin and cyclophosphamide.

A particular combination for use in treating HER2 breast cancer comprises a pharmaceutical formulation of the present invention and lapatinib.

Combinations of pharmaceutical formulations of the present invention with cyclophosphamide, doxorubicin (hydroxydaunorubicin), vincristine, rituximab and prednisone are particularly suitable for treating non Hodgkin's lymphoma (and in particular high grade non Hodgkin's lymphoma).

Combinations of pharmaceutical formulations of the present invention with cyclophosphamide, vincristine, rituximab and prednisone are particularly suitable for treating non Hodgkin's lymphoma (and in particular low grade non Hodgkin's lymphoma).

Particularly suitable for treating multiple myeloma are combinations of pharmaceutical formulations of the present invention with (a) monoclonal antibodies (e.g. those targeting Interleukin 6); (b) proteasome inhibitors (e.g. bortezomib); (c) proteasome inhibitors and corticosteroids (e.g. velcade and dexamethasone); and (d) corticosteroids, alkylating agents and lenolidamide/thalidomide (e.g. prednisolone, melphalan and thalidomide).

Specific combinations suitable for treating multiple myeloma are combinations of pharmaceutical formulations of the present invention with vincristine, doxorubicin, thalidomide and dexamethasone.

Combinations of pharmaceutical formulations of the present invention with fludarabine and rituxamab are particularly suitable for treating chronic lymphocytic leukemia.

Particularly suitable for treating melanoma are combinations of pharmaceutical formulations of the present invention with (a) DNA methylase inhibitors/hypomethylating agents (e.g. temozolamide); (b) alkylating agents (e.g. dacarbazine or fotemustine); and (c) DNA methylase inhibitors/hypomethylating agents (e.g. temozolamide) and DNA repair inhibitors/PARP inhibitors.

Particularly suitable for treating gastrointestinal stromal tumors (GIST) are combinations of pharmaceutical formulations of the present invention with an ancillary agent selected from imatinib, nilotinib, dasatinib and sunitinib.

Particularly suitable for treating prostate cancer are combinations of pharmaceutical formulations of the present invention with hormones and G-protein coupled receptor inhibitors.

Particularly suitable for treating Non Small Cell Lung Cancer (NSCLC) are combinations of pharmaceutical formulations of the present invention with (a) platinum compounds and taxanes; (b) platinum compounds and antimetabolites; (c) gefitinib and/or cetuximab.

One particular combination for use in treating NSCLC comprises a pharmaceutical formulation of the present invention and gefitinib and/or cetuximab.

For cancer (and in particular acute myeloid leukemia) treatment, two or more anti-cancer agents independently selected from two or more of anthracycline, Ara C (a.k.a. Cytarabine), 6-mercaptopurine, thiopurine, methotrexate, mitoxantrone, daunorubicin, idarubicin, gemtuzumab ozogamicin and granulocyte colony stimulating factors may be used in combination with the pharmaceutical formulations of the present invention. Alternatively, the two or more anti-cancer agents may be independently selected from two or more of anthracycline, Ara C (a.k.a. Cytarabine), daunorubicin, idarubicin, gemtuzumab ozogamicin and granulocyte colony stimulating factors.

For cancer (and in particular breast cancer) treatment, two or more anti-cancer agents independently selected from bevacizumab, taxanes, methotrexate, paclitaxel, docetaxel, gemcitabine, anastrozole, exemestane, letrozole, tamoxifen, doxorubicin, herceptin, 5-fluorouracil, cyclophosphamide, epirubicin and capecitabine, particularly 5-FU, methotrexate and cyclophosphamide; 5FU, doxorubicin and cyclophosphamide; or doxorubicin and cyclophosphamide may be used in combination with the pharmaceutical formulations of the present invention. Preferably, for cancer (and in particular breast cancer) treatment, the two or more anti-cancer agents may also be independently selected from taxanes, methotrexate, paclitaxel, docetaxel, gemcitabine, anastrozole, exemestane, letrozole, tamoxifen, doxorubicin, herceptin, 5-fluorouracil, cyclophosphamide, epirubicin and capecitabine, particularly 5-FU, methotrexate and cyclophosphamide; 5FU, doxorubicin and cyclophosphamide; or doxorubicin and cyclophosphamide.

For cancer (and in particular chronic lymphocytic leukemia (CLL)) treatment, two or more anti-cancer agents independently selected from alemtuzumab, chlorambucil, cyclophosphamide, almentuzumab, vincristine, predinisolone, fludarabine, mitoxantrone and rituximab/rituxamab, particularly fludarabine and rituxamab may be used in combination with the pharmaceutical formulations of the present invention. Preferably, for cancer (and in particular chronic lymphocytic leukemia (CLL)) treatment, the two or more anti-cancer agents are independently selected from chlorambucil, cyclophosphamide, vincristine, predinisolone, fludarabine, mitoxantrone and rituximab/rituxamab, particularly fludarabine and rituxamab.

For cancer (and in particular chronic myeloid leukemia (CML)) treatment, two or more anti-cancer agents independently selected from hydroxyurea, cytarabine, desatinib, nilotinib and imatinib may be used in combination with the pharmaceutical formulations of the present invention.

For cancer (and in particular colon cancer treatment), two or more anti-cancer agents independently selected from cetuximab, 5-Fluorouracil, pantumab, leucovorin, irinotecan, oxaliplatin, raltirexed, capecitabine, bevacizumab, oxaliplatin, CPT 11, particularly 5-Fluorouracil, Leucovorin and CPT 11 or Fluorouracil, Leucovorin and Oxaliplatin may be used in combination with the pharmaceutical formulations of the present invention.

Alternatively, for cancer (and in particular colon cancer treatment), two or more anti-cancer agents independently selected from 5-Fluorouracil, leucovorin, irinotecan, oxaliplatin, raltirexed, capecitabine, bevacizumab, oxaliplatin, CPT 11 and particularly 5-Fluorouracil, Leucovorin and CPT 11 or Fluorouracil, Leucovorin and Oxaliplatin may be used in combination with the pharmaceutical formulations of the present invention

For cancer (and in particular multiple myeloma treatment), two or more anti-cancer agents independently selected from vincristine, doxorubicin, dexamethasone, melphalan, prednisone, cyclophosphamide, etoposide, pamidronate, thalidomide, zoledronate and bortezomib, particularly vincristine, doxorubicin and dexamethasone may be used in combination with the pharmaceutical formulations of the present invention.

For cancer (and in particular Non-Hodgkin's lymphoma treatment), two or more anti-cancer agents independently selected from cyclophosphamide, doxorubicin/hydroxydaunorubicin, vincristine/Onco-TCS (V/O), prednisolone, methotrexate, cytarabine, bleomycin, etoposide, rituximab/rituxamab, fludarabine, cisplatin, and ifosphamide, particularly cyclophosphamide, doxorubicin (hydroxydaunorubicin), vincristine and prednisone for high grade NHL or cyclophosphamide, vincristine and prednisone for low grade NHL may be used in combination with the pharmaceutical formulations of the present invention.

For cancer (and in particular Non Small Cell Lung Cancer (NSCLC)) treatment, two or more anti-cancer agents may be independently selected from bevacizumab, gefitinib, erlotinib, cisplatin, carboplatin, mitomycin, vinblastine, paclitaxel, docetaxel, gemcitabine and vinorelbine, especially taxol and carboplatin or gemcitabine and cisplatin may be used in combination with the pharmaceutical formulations of the present invention.

For cancer (and in particular ovarian cancer) treatment, two or more anti-cancer agents independently selected from platinum compounds (for example Cisplatin, Carboplatin), doxorubicin, liposomal doxorubicin, paclitaxel, docetaxel, gemcitabine, melphalan and mitoxantrone may be used in combination with pharmaceutical formulations of the present invention.

For cancer (and in in particular prostate cancer) treatment, two or more anti-cancer agents independently selected from mitoxantrone, prednisone, buserelin, goserelin, bicalutamide, nilutamide, flutamide, cyproterone acetate, megestrol/megestrel, diethylstilboestrol, docetaxel, paclitaxel, zoledronic acid, prednisolone and taxotere may be used in combination with the pharmaceutical formulations of the present invention.

In a particularly preferred embodiment, the pharmaceutical formulation of the present invention is administered in combination with one or more ancillary agents selected from cisplatin, bortezomib, erlotinib, paclitaxel, trastuzumab and cytarabine.

Where the formulations of the invention are used in combination with ancillary compounds or with other therapies, the two or more treatments may be given in individually varying dose schedules and via different routes.

Where the pharmaceutical formulations of the present invention are administered in combination therapy with one, two, three, four or more other therapeutic agents (preferably one or two, more preferably one), the compounds may be administered simultaneously or sequentially. When administered sequentially, they may be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The pharmaceutical formulations of the present invention also be administered in conjunction with non-chemotherapeutic treatments such as radiotherapy, photodynamic therapy, gene therapy; surgery and controlled diets.

For use in combination therapy with another chemotherapeutic agent, the pharmaceutical formulations of the present invention and one, two, three, four or more other therapeutic agents may be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through his or her common general knowledge the dosing regimes and combination therapies to use.

In further aspects of the invention, there are provided:
a combination (for example for use in treating non-small cell lung cancer) comprising a pharmaceutical formulation of the present invention and gefitinib and/or cetuximab;
a combination (for example for use in treating gastrointestinal stromal tumors (GIST)) comprising a pharmaceutical formulation of the present invention and an ancillary agent selected from imatinib, nilotinib, dasatinib and sunitinib;
a combination (for example for use in treating HER2 breast cancer) comprising a pharmaceutical formulation of the present invention and lapatinib; and
a combination (for example for use in treating acute myeloid leukaemia) comprising a pharmaceutical formulation of the present invention and an ancillary agent selected from daunorubicin and idarubicin.

The invention will now be further described in the Examples below, which are intended as an illustration only and do not limit the scope of the invention.

### EXAMPLES

### Example 1

### Synthesis of (2,4-dihydroxy-5-isopropyl-phenyl)-[5-(4-methyl-piperazin-1-ylmethyl)-1,3-dihydro-isoindol-2-yl]-methanone (Compound of Formula (I))

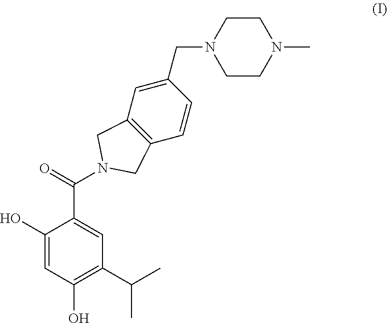 [SMILES: CC(C)c1cc(C(=O)N2Cc3ccc(CN4CCN(C)CC4)cc3C2)c(O)cc1O]

The synthesis of the compound of formula (I) is found in U.S. Pat. No. 7,700,625.

### A. Synthesis of 2-(2,4-bis-benzyloxy-5-isopropyl-benzoyl)-2,3-dihydro-1H-isoindole-5-carboxylic acid

A solution of 2-(2,4-bis-benzyloxy-5-isopropyl-benzoyl)-2,3-dihydro-1H-isoindole-5-carboxylic acid methyl ester (390 mg) in methanol (10 ml) and 2M NaOH (10 ml) was heated at 50° C. for 48 hours then evaporated. The residue was acidified with 2M HCl, the solid collected by filtration, washed with water and sucked dry to give 255 mg of 2-(2,4-bis-benzyloxy-5-isopropyl-benzoyl)-2,3-dihydro-1H-isoindole-5-carboxylic acid as a white solid. [M+H]⁺ 520.

### B. Synthesis of 2-(2,4-bis-benzyloxy-5-isopropyl-benzoyl)-2,3-dihydro-1H-isoindole-5-carboxylic acid methoxy-methyl-amide

A solution of 2-(2,4-bis-benzyloxy-5-isopropyl-benzoyl)-2,3-dihydro-1H-isoindole-5-carboxylic acid (1.76 g, 3.39 mmol), EDC (0.78 g, 4.06 mmol), HOBT (0.55 g, 4.06 mmol), Et₃N (1 ml, 6.78 mmol) and N,O-dimethylhydroxylamine hydrochloride (0.36 g, 3.72 mmol) in DMF (20 ml) was stirred at room temperature for 48 hours, then evaporated under vacuum. The crude material was dissolved in ethyl acetate and extracted twice with saturated NaHCO₃, organics washed with brine, dried (MgSO₄), filtered then evaporated to give 1.84 g of 2-(2,4-bis-benzyloxy-5-isopropyl-benzoyl)-2,3-dihydro-1H-isoindole-5-carboxylic acid methoxy-methyl-amide. MS: [M+H]⁺ 563.

### C. Synthesis of 2-(2,4-bis-benzyloxy-5-isopropyl-benzoyl)-2,3-dihydro-1H-isoindole-5-carbaldehyde

A solution of 2-(2,4-bis-benzyloxy-5-isopropyl-benzoyl)-2,3-dihydro-1H-isoindole-5-carboxylic acid methoxy-methyl-amide (0.226 g, 0.4 mmol) in THF (5 ml) cooled to 0° C., treated with 1M LiAlH₄/THF (0.3 ml, 0.3 mmol), stirred 1 hour, further LiAlH₄ (0.05 ml) added then stirred for 30 minutes. The reaction was quenched with saturated KHSO₄ solution, extracted with EtOAc, dried (MgSO₄), filtered and evaporated to give 0.2 g of 2-(2,4-bis-benzyloxy-5-isopropyl-benzoyl)-2,3-dihydro-1H-isoindo-le-5-carbaldehyde. MS: [M+H]⁺ 504.

### D. Synthesis of (2,4-bis-benzyloxy-5-isopropyl-phenyl)-[5-(4-methyl-piperazin-1-ylmethyl)-1,3-dihydro-isoindol-2-yl]-methanone

To a solution of 2-(2,4-bis-benzyloxy-5-isopropyl-benzoyl)-2,3-dihydro-1H-isoindole-5-carbaldehyde (0.316 g, 0.63 mmol) and n-methyl piperazine (63 mg 0.63 mmol) in CH₂Cl₂ (10 ml) was added AcOH (38 mgs 0.63 mmol) and NaBH(OAc)₃ (0.28 g, 1.33 mmol), then stirred at ambient for 5 hours. The reaction was quenched with water, layers separated and aqueous washed CH₂Cl₂. The organics were combined, washed with brine, dried (MgSO₄), filtered and evaporated to give 0.32 g of (2,4-bis-benzyloxy-5-isopropyl-phenyl)-[5-(4-methyl-piperazin-1-ylmethyl)-1,3-dihydro-isoindol-2-yl]-methanone. MS: [M+H]⁺ 588.

### E. Synthesis of (2,4-dihydroxy-5-isopropyl-phenyl)-[5-(4-methyl-piperazin-1-ylmethyl)-1,3-dihydro-isoindol-2-yl]-methanone

Hydrogenation was carried out using a stirred solution of protected derivative (1 equivalent) and a catalytic amount of 10% palladium on carbon (typically 30-50 mg) in ethanol (5-10 ml), methanol (5-10 ml) or methanol/DCM (3 ml/3 ml was stirred at room temperature under an atmosphere of hydrogen for 2-16 hours. K₂CO₃ (2 equiv.) in a MeOH/H₂O [9.1] was added. The catalyst was removed by filtration, washed with methanol (5 ml) and the solvent removed in vacuo to afford the products. Some required purification by flash chromatography, eluting typically with ether. After evaporation of methanol the reaction was diluted with water, neutralized using 1M HCl and extracted with CH₂Cl₂ (×2). Organics dried (MgSO₄), filtered and evaporated under vacuum then purified by preparative HPLC to give 21 mg of (2,4-dihydroxy-5-isopropyl-phenyl)-[5-(4-methyl-piperazin-1-ylmethyl)-1,3-dihydro-isoindol-2-yl]-methanone. MS: [M+H]⁻ 410. ¹H NMR (Me-d₃-OD) 7.37-7.23 (3H, br s), 7.19 (1H, s), 6.39 (1H, s), 4.94-4.87 (4H, br s), 3.57 (2H, s), 3.27-3.16 (1H, m), 2.67-2.39 (8H, m), 2.31 (3H, s), 1.23 (6H, d).

### Example 2

### Preparation of the L-Lactate Salt of the Compound of Formula (I)

U.S. Published Application Serial No. 2011-0046155 discloses the preparation of the L-lactate salt of (2,4-dihydroxy-5-isopropyl-phenyl)-[5-(4-methyl-piperazin-1-ylmethyl)-1,3-dihydro-isoindol-2-yl]-methanone and the procedure was followed herein. The product of Example 1 (1.24 g, 3.303 mmol) was suspended in ethanol (3 mL) and EtOAc (5 mL) and a solution of L-lactic acid (0.285 g, 3.13 mmol) dissolved in ethanol (3 mL) was added. The solution was heated until clear and then was filtered. EtOAc (5 mL) was used to wash the filter and the combined filtrates were stirred at RT for 2 h with seeding. The crystalline mass which formed was removed by filtration, was washed with EtOAc and then dried in vacuum at 50° C. to give the title compound 1.29 g. 1H NMR (400 MHz, Me-d3-OD): 7.30 (s, 3H), 7.18 (s, 1H), 6.39 (s, 1H), 4.91 (s, 4H), 4.08 (q, J=6.8 Hz, 1H), 3.70-3.63 (m, 2H), 3.28-3.15 (m, 1H), 3.01 (s, 4H), 2.68 (m, 7H), 1.36 (d, J=6.8 Hz, 3H), 1.23 (d, J=6.9 Hz, 6H).

### Example 2A

### (2,4-Dihydroxy-5-isopropyl-phenyl)-[5-(4-methyl-piperazin-1-ylmethyl)-1,3-dihydro-isoindol-2-yl]-methanone L-lactate salt

Example 2A describes a synthetic route containing essentially the same process steps as the route described in Examples 1 and 2 but wherein the process conditions are more suited to larger scale reactions.

Step 1

### 4-Acetoxy-2-hydroxy-benzoic acid methyl ester

To a heated solution (50° C.) of resorcinol methyl ester (16.5 Kg, 98.1 mol) and N,N-dimethyl-4-aminopyridine (89.1 g, 0.73 mol, 7.4 mol %) in toluene (66 L) was slowly added (over 2 h) acetic anhydride (9.9 L, 104.9 mol). The solution was heated to 50° C. for a further 1.5 h and then the solvent was removed by evaporation at 50° C. to a small volume and the residue was azeotroped once with toluene. To the residual oil was immediately added toluene (33 L) whilst still warm and the solution used for Step 2 without further purification.

Step 2

### 5-Acetyl-2,4-dihydroxy-benzoic acid methyl ester

The toluene solution from Step 1 was cooled in an ice bath under N₂ and triflic acid (9.44 L) added slowly over 3 h. On stirring a fine white solid was formed which dissolved on warming to RT over 20 h and then stirring at RT for 37 h to give a yellow solution. To the solution was added acetyl chloride (726 mL) and the solution stirred at RT for a further 1 h. This solution was cannulated into a stirred cooled (0° C.) solution of EtOAc (217.8 L) and NaOAc₃H₂O (14.52 Kg) dissolved in water (145 L). The organic phase was washed with saturated brine (twice, 72.6 L), and was evaporated to 5.5 Kg. Toluene:isopropanol (2:3) was added and the crystalline solid removed by filtration and dried to give 12.6 Kg (61% over 2 steps), mp 124-126° C.

Step 3

### 5-Acetyl-2,4-bis-benzyloxy-benzoic acid methyl ester

To a stirred solution of benzyl bromide (16.14 L, 136 mol) and anhydrous potassium carbonate (20.25 Kg, 147.6 mol) in acetonitrile (184.5 L) was added methyl 5-acetyl-2,4-dihydroxybenzoate (14 Kg, 66.6 mol, step 2) in 6 portions over 5 h. The mixture was stirred and held at reflux for 20 hours, cooled to room temperature the mixture was poured onto water (682 L) and stirred vigorously for 2 hours. The solids were collected by centrifugation and dried under reduced pressure to constant mass in a vacuum oven at 60° C. overnight to afford methyl 5-acetyl-2,4-bis-benzyloxybenzoate (23.5 Kg, 97.3%) as a cream solid mp 114-115° C.

Step 4

### 2,4-Bis-benzyloxy-5-isopropenyl-benzoic acid methyl ester

A solution of potassium tert-butoxide (6.72 Kg, 60.1 mol) in anhydrous THF (60 L) was added over 3 h to a stirred suspension of methyltriphenylphosphonium bromide (21.43 Kg, 60.1 mol) and methyl 5-acetyl-2,4-bis-benzyloxybenzoate (21.3 Kg, 54.6 mol, step 3) in anhydrous tetrahydrofuran (213 L) at 15° C. The mixture was stirred at 15° C. for 70 mins and the warmed to 20° C. over 60 mins. Methanol (27.3 L) was added to quench excess phosphorus ylide and the solvent was concentrated in vacuo followed by addition of EtOAc and water. The organic phase was treated with activated charcoal, filtered and evaporated to a small volume. The residue was crystallized from boiling MeOH and the solids were collected by suction filtration, washed with methanol and dried under reduced pressure to afford methyl 2,4-bis-benzyloxy-5-isopropenyl-benzoate 18.1 Kg (85%) as pale yellow needles mp 92-94° C. (99.6% pure by hplc).

Step 5

### 2,4-Bis-benzyloxy-5-isopropenyl-benzoic acid

Potassium hydroxide (0.527 Kg, 9.4 mol) was added to a stirred suspension of methyl 2,4-bis-benzyloxy-5-isopropenyl-benzoate (3.1 Kg, 8 mol, step 4) in methanol (18.6 L) and water (12.4 L) and the mixture was stirred and held at reflux for 3 hours. The methanol was removed under partial vacuum from the vessel, and to the remaining solution was added toluene (62 L). The solution was heated to 40° C. and to the mixture was added conc HCl (1.36 L). The biphasic mixture was heated to 50° C. and the phases separated. The organic phase was washed with water (31 L) at 50° C. and the organic phase was evaporated under reduced pressure to give 2,4-bis-benzyloxy-5-isopropenyl-benzoic acid 2.851 Kg (95% yield) as a colorless solid.

Step 6

### Di-prop-2-ynyl-carbamic acid benzyl ester

To a cooled (5° C.) solution of K₂CO₃ (4 Kg, 29.0 mol) in water (17.5 L) and toluene (12.5 L) was added dipropargylamine (2.50 Kg, 26.88 mol). Benzyloxychloroformate (4.8 Kg, 28.14 mol) was added at a rate such that T<10° C. The solution was stirred at 5° C. for 10 mins and then allowed to warm to RT. The aqueous phase was separated and the organic phase was washed with 0.2M HCl (12.5 L), sat NaHCO₃ (13.5 L) and brine (17 L) and the resultant solution used in step 7 (assayed to contain 6.23 Kg, 102% based on an evaporated portion).

Step 7

### 5-Hydroxymethyl-1,3-dihydro-isoindole-2-carboxylic acid benzyl ester

A solution of propargyl alcohol (2.11 Kg, 37.7 mol) in toluene (32.48 L) was degassed and heated to 55° C. The solution of di-prop-2-ynyl-carbamic acid benzyl ester (4.06 Kg, 17.86 mol, step 6) in toluene and Wilkinsons catalyst (0.162 Kg) were added in 10 equal portions such that temperature <65° C. (the exotherm was allowed to subside before the next addition was made). The solution was then stirred at 55° C. for 1 h and then cooled to 20° C. DCM (8.12 L) was added and the mixture was concentrated to a small volume. Toluene (8 L) was added and the solution evaporated to constant weight giving the title compound 5.72 Kg (113%).

Step 8

### 5-Methanesulfonyloxymethyl-1,3-dihydro-isoindole-2-carboxylic acid benzyl ester

To a cooled solution (5° C.) of 5-hydroxymethyl-1,3-dihydro-isoindole-2-carboxylic acid benzyl ester (11 Kg, 38.8 mol, step 7) and Et₃N (7.04 L, 50.6 mol) in DCM (55 L) was added methanesulphonyl chloride (2.97 L, 38.4 mol) so that the internal temp <10° C. After stirring for 0.5 h at 5° C., the solution was used below in step 9.

Step 9

### 5-(4-Methyl-piperazin-1-ylmethyl)-1,3-dihydro-isoindole-2-carboxylic acid benzyl ester dihydrochloride salt

The solid from Step 8 (0.232 mol) was dissolved in acetone (700 mL) and this solution was added over 45 mins to a cooled (internal temp 15-17° C.) suspension of K₂CO₃ (48 g) and N-methylpiperazine (50 mL, 0.45 mol) in acetone (330 mL). The suspension was stirred at 15° C. for 3 h (complete removal of starting material by tic) when the solution was evaporated to a small volume and the residue partition between EtOAc (1000 mL) and a mixture of water (500 mL) and saturated brine (50 mL). The organic phase was washed with a mixture of water (500 mL) and saturated brine (150 mL) and finally washed with saturated brine (300 mL). The solution was dried (MgSO₄) and filtered and to this solution was added 1 M-HCl in MeOH (430 mL, 0.43 mol). The suspension was cooled (0° C. for 30 mins) and the solid removed by filtration which was washed with EtOAc and then heptane on the sinter and the solid dried (oil-pump, RT 72 h) to give crop 1 of the title compound 66.34 g (65%) as a colorless solid. ¹H NMR (400 MHz, Me-d3-OD): 7.64-7.51 (m, 2H), 7.51-7.29 (m, 6H), 5.23 (s, 2H), 4.79 (dd, J=16.2, 6.1 Hz, 4H), 4.49 (s, 2H), 3.66 (s, 8H), 3.03 (s, 3H).

Step 10

### 5-(4-Methyl-piperazin-1-ylmethyl)-1,3-dihydro-isoindole-2-carboxylic acid benzyl ester

DCM (33 L) and N-methylpiperazine (21.45 L, 193.4 mol) were stirred at 25° C. and the solution from step 9 added over a minimum of 30 mins such that temperature 20-30° C. After stirring the solution for a further 30 mins water (55 L) was added and the organic phase was washed with water (2×55 L). The product was extracted into 0.8M HCl (66 L) and the layers separated. The aqueous phase was washed with DCM (55 L) and then basified with 2M NaOH to pH 10-11 and the product was extracted into EtOAc (2×55 L). The combined organic phase were filtered to remove solids and the evaporated followed by azeotroping with toluene and drying to constant weight to give the title compound, 6.63 kg (47% yield, 98% pure by hplc).

Step 11

### 5-(4-Methyl-piperazin-1-ylmethyl)-2,3-dihydro-1H-isoindole

To a degassed solution of 5-(4-Methyl-piperazin-1-ylmethyl)-1,3-dihydro-isoindole-2-carboxylic acid benzyl ester (Step 10, 1.3 Kg, 3.55 mol) dissolved in EtOH (13 L) was added 10% Pd/C (0.065 Kg). Hydrogen was passed through the mixture at 30° C. for 4 h or until complete by NMR. The solution was then stirred for 1 h under an atmosphere of N₂ and then filtered to remove the catalyst through a GF/F filter followed by filtration through a Cuno filter. The filtrate was evaporated to a small volume, azeotroped with toluene (3.9 L) and dried to constant weight yielding the title compound as a red/black oily solid (0.78 Kg) which was stored under nitrogen until required.

Step 12

### (2,4-Bis-benzyloxy-5-isopropenyl-phenyl)-[5-(4-methyl-piperazin-1-ylmethyl-)-1,3-dihydro-isoindol-2-yl]-methanone

1,1′-Carbonyldiimidazole (4.82 Kg, 29.8 mol) was added to a solution of 2,4-bis-benzyloxy-5-isopropenyl-benzoic acid (10.58 Kg, 28.3 mol, step 5) in DMF (21.2 L) at 25° C. After 20 mins at 25° C. a solution of 5-(4-Methyl-piperazin-1-ylmethyl)-2,3-dihydro-1H-isoindole (7.2 Kg, 31.1 mol, step 10) in DMF (7.2 L) maintaining a temperature below 35° C. and the solution stirred at 25° C. for a minimum of 12 h. The solid which had formed was removed by filtration, washed with isopropyl acetate (2×21.6 L) and dried at 35° C. to constant weight to give the title compound 8.7 Kg (77% yield, purity by hplc 97.5%).

Step 13

### (2,4-Dihydroxy-5-isopropyl-phenyl)-[5-(4-methyl-piperazin-1-ylmethyl)-1,3-dihydro-isoindol-2-yl]-methanone

The product from Step 12 (0.9 Kg, 1.53 mol) was dissolved in isopropanol (6.8 L) and water (1.04 L) and after purging with N₂ 10% Pd/C (90 g) and K₂CO₃ (0.212 Kg, 1.53 mol) were added and the suspension was hydrogenated for 60 to 70 mins under a 3 Barr pressure of H₂. The solution was diluted with water (0.5 L) and filtered. To the filtrate was added aqueous HCl (30% hydrochloric acid, 0.85 Kg diluted with water 5.42 Kg) and the solution was concentrated at 60° C. under vacuum (removing 10 L isopropanol). Water (0.45 L) was added to the solution and concentration continued (until a further 10 L isopropanol had been removed). The aqueous phase was washed with EtOAc (4.61 L), diluted with acetonitrile (4.06 L) and neutralized to pH 7.5-8.5 by addition of conc ammonia solution (0.35 Kg). The suspension was stirred for 2.5 h and then the solid was removed by filtration. The residue was washed with acetonitrile (2×0.8 L) and dried at 40° C. to constant weight to give the title compound 588 g (94% yield).

Step 14

### (2,4-Dihydroxy-5-isopropyl-phenyl)-[5-(4-methyl-piperazin-1-ylmethyl)-1,3-dihydro-isoindol-2-yl]-methanone L-lactate salt (form FL1)

The product of Step 13 (646 g, 1.58 mol) was dissolved in ethanol (5.17 L) and the solution filtered. A solution of L-lactic acid (142 g, 1.58 mol) dissolved in ethanol (2.59 L) was filtered and added to the solution of the filtered solution (above) and then to the mixture was added EtOAc (7.75 L). The suspension was stirred at RT for 12 h and then cooled to 5° C. for a further 2 h. The solid which had formed was removed by filtration, washed with EtOAc (2×2.58 L) and heptane (2×1.94 L) and dried to constant weight at 35° C. giving the title compound (581 g, 74% yield).

### Example 3

### Formulations of L-Lactate Salt of Compound of Formula (I) with Phosphate Buffers

Buffers were prepared using monobasic and dibasic salts of sodium phosphate in order to achieve the correct buffer concentrations and pH as follows:

| Buffer | 50 mM | | 100 mM | | 200 mM | |
|---|---|---|---|---|---|---|
| PH/ | Sodium | Sodium | Sodium | Sodium | Sodium | Sodium |
| Buffer | Phosphate | Phosphate | Phosphate | Phosphate | Phosphate | Phosphate |
| Conc. | (Monobasic) | (Dibasic) | (Monobasic) | (Dibasic) | (Monobasic) | (Dibasic) |
| 5.0 | 6.807 g/l | 0.095 g/l | 13.615 g/l | 0.191 g/l | 27.229 g/l | 0.382 g/l |
| 5.5 | 6.615 g/l | 0.293 g/l | 13.229 g/l | 0.587 g/l | 26.459 g/l | 1.174 g/l |

Note:
The table above used weights of monosodium phosphate, monohydrate (MW 137.99) and Disodium phosphate, anhydrous (MW 141.96).

The pH of each buffer was the target pH±0.05. Minor adjustments with small amounts of dilute sodium hydroxide were made to both of the 200 mM buffers to meet this criterion.

Aliquots of 1 g of the L-lactate salt of the compound of formula (I) were weighed into individual 20 ml scintillation vials and 10 ml of each buffer was added to a vial. An additional sample was prepared in deionised water. Samples were covered in aluminium foil to protect from light and mixed using a magnetic stirrer overnight.

The appearance of each sample was recorded. Samples were transferred to centrifuge tubes and centrifuged at 3500rpm for 15 minutes. The samples prepared using the 200 mM buffers were visually free of undissolved material, but were centrifuged in order to make sure that all samples were treated the same. The supernatants were retained for determination of compound of formula (I) by UV and pH measurement.

Assay by UV: A calibration curve was prepared using the L-lactate salt of the compound of formula (I) dissolved in deionised water and covering the range 0 to 0.10 mg/ml. Absorbance was determined at 286 nm. Samples were diluted as necessary using deionised water and absorbance determined at 286 nm versus a blank. The content of the compound of formula (I) was determined using the standard curve. All UV readings were taken in duplicate.

Results

| | L-Lactate Salt of | |
|---|---|---|
| Sodium Phosphate | Compound of Formula (I) | pH of |
| Buffer | Concentration (mg/ml) | Supernatant |
| 50 mM/pH 5.0 | 77.3 | 5.13 |
| 50 mM/pH 5.5 | 75.9 | 5.18 |
| 200 mM/pH 5.0 | 89.0 | 5.05 |
| 100 mM/pH 5.5 | 86.3 | 5.10 |
| 200 mM/pH 5.0 | 95.7 | 4.99 |
| 200 mM/pH 5.5 | 94.8 | 5.08 |

The 200 mM sodium phosphate buffer with a nominal pH of 5.0 offered the best solubility.

### Example 4

### Formulation for Intravenous Administration

Following the procedures in the Examples outlined above, the following formulation suitable for intravenous administration was prepared:

| Component/ | Quantity/ | | Quantity/ | | Quantity/ | |
|---|---|---|---|---|---|---|
| Excipient | mL | | vial | | lot | |
| L-Lactate Salt | 50.0 | mg (*) | 265.00 | mg (*) | 5.00 | g (*) |
| of compound of | | | | | | |
| formula (I) | | | | | | |
| Sodium dihydrogen | 25.59 | mg | 135.63 | mg | 2.56 | g |
| phosphate | | | | | | |
| monohydrate | | | | | | |
| Di-sodium hydrogen | 2.28 | mg | 12.08 | mg | 228 | mg |
| phosphate | | | | | | |
| dihydrate | | | | | | |

| HCl (1M) | q.s. to pH 5.0 ± 0.1 | | |
|---|---|---|---|
| | (if needed) | | |
| NaOH (1M) | q.s. to pH 5.0 ± 0.1 | | |
| | (if needed) | | |
| Water for | qs to 1.0 mL | qs to 5.3 mL | Q.S .to 100 mL |
| injection | | | |

### Example 5

### Formulations of L-Lactate Salt of Compound of Formula (I) with Other Buffers Compared to Formulation with Phosphate Buffer

In a manner analogous to Example 3, formulations were prepared of the L-lactate salt of the compound of formula (I) with acetate, succinate and citrate buffers at different concentrations and at different pH. As shown in the table below, neither the acetate nor citrate buffers were as useful as the phosphate buffer formulation of Example 3, but the succinate buffer gave good L-lactate salt solubility.

| | | | Solubility |
|---|---|---|---|
| | | | L-lactate salt of |
| | Appearance Prior to | | compound of |
| Buffer | Centrifugation | pH | formula (I) (mg/ml) |
| Deionised water | Off-white | 5.52 | 59.45 |
| | suspension/liquid | | |
| 50 mM | Viscous suspension | 4.87 | 24.35 |
| Acetate pH 5.5 | | | |
| 50 mM | Off-white | 5.03 | 19.12 |
| Acetate pH 6.0 | suspension/liquid, | | |
| | Possibly more viscous | | |
| | then water sample | | |
| 100 mM | Off-white solid | NT | NT |
| Acetate pH 5.5 | | | |
| 100 mM | Off-white solid | NT | NT |
| Acetate pH 6.0 | | | |
| 50 mM | Off-white | 5.38 | 78.30 |
| Succinate pH 5.5 | suspension/liquid | | |
| 50 mM | Off-white | 5.69 | 78.29 |
| Succinate pH 6.0 | suspension/liquid | | |
| 100 mM | Off-white | 5.30 | 89.23 |
| Succinate pH 5.5 | suspension/liquid | | |
| 100 mM | Off-white | 5.76 | 89.88 |
| Succinate pH 6.0 | suspension/liquid | | |
| 50 mM | Off-white | 5.23 | 85.61 |
| Citrate pH 5.5 | suspension/liquid | | |
| 50 mM | Off-white | 5.55 | 83.94 |
| Citrate pH 6.0 | suspension/liquid | | |
| 100 mM | Solid cake with | NT | NT |
| Citrate pH 5.5 | clear liquid on top | | |
| 100 mM | Solid cake with | NT | NT |
| Citrate pH 6.0 | clear liquid on top | | |

NT = Not Tested

### Example 6

### The Effect of Surfactant on Solubility

Solutions of sodium phosphate buffer at a pH of 7.2 were prepared with and without polysorbate 80 (Tween) and the solubilities of the L-lactate salt of the compound of formula (I) were determined. The results are shown in the table below.

| | | | Solubility of L- |
|---|---|---|---|
| | Concentration of | | lactate salt of |
| | Tween - % (w/v) of | pH of | compound of |
| Buffer | solution | Solution | formula (I) (mg/ml) |
| 200 mM phosphate | 0 | 7.2 | 21 |
| 200 mM phosphate | 1-8 | 7.2 | 33-34 |

The data show that, at physiological pH, the addition of the non-ionic surfactant “Tween” results in a 1.5 fold increase in the solubility of the L-lactate salt.

### Example 7

### Biological Activity

(2,4-Dihydroxy-5-isopropyl-phenyl)-[5-(4-methyl-piperazin-1-ylmethyl)-1,3-dihydro-isoindol-2-yl]-methanone (AT13387) is a potent HSP90 inhibitor and has potent anticancer activity against a range of cancer types in both in vitro and in vivo assays. As a result of its anti-cancer activity, AT13387 is in clinical trials against a range of cancers including refractory solid tumours, melanoma and imatinib-resistant GIST.

In a Phase I study, AT13387 was found to be well tolerated by patients with advanced solid tumours subjected to a Two-Consecutive Day (QD×2) dosing schedule.

In another Phase I study, AT13387 was shown to be effective in both vemurafenib sensitive and resistant models of melanoma (Rodriguez-Lopez et al., AACR Poster, 2012—Abstract 2772).

Shapiro et al., “Heat Shock Protein Inhibitor in Patients With Refractory Solid Tumours: A Phase I Pharmacokinetic and Pharmacodynamic Study Of AT13387”, ASCO Annual Meeting (2010) Poster presentation, describes Phase I clinical studies carried out on a group of twenty one patients (later expanded to twenty six patients) suffering from a variety of solid tumours which were refractory to standard therapy. In the clinical study, twenty six patients (15 female and 11 male) with histologically confirmed solid tumours received (2,4-dihydroxy-5-isopropyl-phenyl)-[5-(4-methyl-piperazin-1-ylmethyl)-1,3-dihydro-isoindol-2-yl]-methanone on a twice weekly schedule.). Following treatment, stable disease for at least six months was observed in two of the original twenty one patients (one with melanoma and one with thyroid cancer) and stable disease for beyond 2 cycles of treatment was observed in a further five of the original twenty one patients. Bearing in mind that the patients in this study had already been treated using other therapies which had not worked, and that consequently the cancers were very well advanced and patient prognoses were very poor, the results described were highly encouraging.

Geoffrey Shapiro (presentation entitled “AT13387 (HSP 90 Inhibitor) given at the conference “111 Annual Targeted Therapies of the Treatment of Lung Cancer” in Santa Monica, Calif. in February 2011) describes the Phase I clinical studies referred to in Shapiro et al. above but includes details of a Gastrointestinal Stromal Tumour (GIST) patient subsequently recruited to the study. Positron emission tomography (PET) scans of the patient suffering from GIST were taken before and after treatment with AT13387 (220 mg/m² weekly). The PET scans demonstrated substantial reduction in the metabolic activity of the tumour following treatment.

Mahadevan et al. (ASCO Annual Meeting 2012—Poster—Abstract 3028—describes Phase 1 clinical studies on AT13387 in refractory solid tumours. The results of the studies showed that objective and durable partial response and stable disease were observed in five patients, including three GIST patients.

Mahadevan et al., Gastrointestinal Cancers Symposium, Jan. 24-26, 2013, in San Francisco, describes a Phase 2 study of the combination of the pharmaceutical formulation of the invention with imatinib for imatinib-resistant GIST.

Smyth et al., (presentation at the World Melanoma 2013 conference in Hamburg, Germany, on Jul. 17-20, 2013) discloses that continuous dual treatment with the pharmaceutical formulation of the invention and vemurafenib delayed the emergence of resistant tumors in the BRAF model and that a number of Phase 2 clinical trials are ongoing in combination with targeted therapy. The data additionally indicated that monotherapy with the pharmaceutical formulation of the invention can also be effective on tumors that have relapsed or progressed during treatment with BRAF inhibitors.

Thus, by inhibiting Hsp90, (2,4-dihydroxy-5-isopropyl-phenyl)-[5-(4-methyl-piperazin-1-ylmethyl)-1,3-dihydro-isoindol-2-yl]-methanone suppresses or reduces levels of oncogenic client proteins of Hsp90 and thereby inhibit or reduce the growth of cancer cells. Pharmaceutical formulations comprising (2,4-dihydroxy-5-isopropyl-phenyl)-[5-(4-methyl-piperazin-1-ylmethyl)-1,3-dihydro-isoindol-2-yl]-methanone, or its L-lactate salt, of the invention, therefore, have good anticancer activity.

It is to be understood that the invention is not limited to the particular embodiments of the invention described above, as variations of the particular embodiments may be made and still fall within the scope of the appended claims.

1. A pharmaceutical formulation, comprising (2,4-dihydroxy-5-isopropyl-phenyl)-[5-(4-methyl-piperazin-1-ylmethyl)-1,3-dihydro-isoindol-2-yl]-methanone of formula (I): 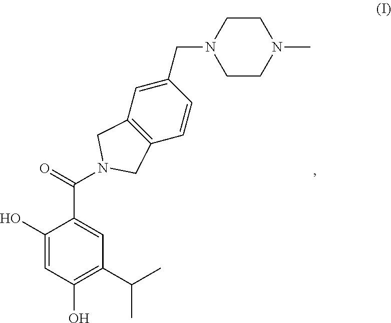 [SMILES: CC(C)c1cc(C(=O)N2Cc3ccc(CN4CCN(C)CC4)cc3C2)c(O)cc1O]
or a L-lactate salt thereof, and a phosphate or succinate buffer.

2. A pharmaceutical formulation according to claim 1 wherein the buffer is a phosphate buffer, for example a sodium phosphate buffer.

3. (canceled)

4. The pharmaceutical formulation according to claim 2, wherein the phosphate buffer is selected from sodium dihydrogen phosphate, disodium hydrogen phosphate and mixtures thereof.

5. The pharmaceutical formulation according to claim 4, wherein the phosphate buffer is sodium dihydrogen phosphate monohydrate.

6. The pharmaceutical formulation according to claim 4, wherein the phosphate buffer is di-sodium hydrogen phosphate dihydrate.

7. The pharmaceutical formulation according to claim 2, wherein the phosphate buffer is a combination of more than one sodium phosphate buffer.

8. The pharmaceutical formulation according to claim 7, wherein the phosphate buffer is a combination of two sodium phosphate buffers, for example, one sodium phosphate buffer is sodium dihydrogen phosphate monohydrate and the other is di-sodium hydrogen phosphate dihydrat.

9. (canceled)

10. The pharmaceutical formulation of claim 1 which is in liquid form.

11. The pharmaceutical formulation of claim 1 which is in solid form.

12. The pharmaceutical formulation of claim 11 which is in the form of a lyophilised powder.

13. The pharmaceutical formulation according to claim 1 which, when in liquid form, or when added to a liquid carrier to give a liquid form, is at a pH of about 4.8 to about 5.4, for example, about 4.8 to about 5.2.

14. (canceled)

15. The pharmaceutical formulation according to claim 2 wherein, when said formulation is in liquid form or when added to a carrier to give a liquid form, said phosphate buffer is at a concentration in the liquid form of about 50 mM to about 250 mM, for example, at a concentration of (i) about 50 mM, or (ii) about 100 mM, or (iii) about 200 mM.

16. (canceled)

17. A liquid pharmaceutical formulation suitable for lyophilization to give a reconstitutable powder, wherein the formulation comprises an aqueous solution containing:
about 48 mg/mL to about 52 mg/mL (e.g. about 50 mg/mL) (free base equivalent) of the L-lactate salt of (2,4-dihydroxy-5-isopropyl-phenyl)-[5-(4-methyl-piperazin-1-ylmethyl)-1,3-dihydro-isoindol-2-yl]-methanone;
about 24 mg/mL to about 27 mg/mL (e.g. about 25.59 mg/mL) of sodium dihydrogen phosphate monohydrate (or an equivalent amount of the anhydrous or dihydrate forms); and
about 1.75 mg/mL to about 2.75 mg/mL (e.g. about 2.28 mg/mL) of di-sodium hydrogen phosphate dihydrate (or an equivalent amount of the anhydrous or monohydrate forms).

18. A pharmaceutical formulation in dry lyophilised form comprising (2,4-dihydroxy-5-isopropyl-phenyl)-[5-(4-methyl-piperazin-1-ylmethyl)-1,3-dihydro-isoindol-2-yl]-methanone L-lactate salt and a phosphate buffer (e.g. a sodium phosphate buffer as defined herein), the phosphate buffer being present in an amount such that when the formulation is reconstituted in an aqueous liquid carrier for injection or infusion to give a solution containing a concentration of 40 mg/ml to 60 mg/ml (e.g. approximately 50 mg/ml) of (2,4-dihydroxy-5-isopropyl-phenyl)-[5-(4-methyl-piperazin-1-ylmethyl)-1,3-dihydro-isoindol-2-yl]-methanone L-lactate salt, the solution has a pH in the range from about 4.6 to about 5.4 (for example about 4.8 to about 5.2).

19. The pharmaceutical formulation of claims 1 which includes one or more auxiliary excipients selected from surfactants, for example polyoxyethylene sorbitan monooleate (polysorbate 80), emulsifiers and cyclodextrins.

20. (canceled)

21. A method of preparing a pharmaceutical formulation as defined in claim 1 in lyophilised form, which method comprises forming a solution of (2,4-dihydroxy-5-isopropyl-phenyl)-[5-(4-methyl-piperazin-1-ylmethyl)-1,3-dihydro-isoindol-2-yl]-methanone L-lactate salt in an aqueous carrier containing a phosphate buffer or succinate buffer as defined herein, wherein the solution has a pH in the range from about 4.6 to about 5.4, for example about 4.8 to about 5.2, and then lyophilising the solution.

22. The pharmaceutical formulation according to claim 1, further comprising an ancillary compound.

23. A combination comprising a pharmaceutical formulation as defined in claim 1, and a further therapeutic agent.

24. A method for treating cancer, comprising the step of administering a therapeutically effective amount of the pharmaceutical formulation of claim 1, to a patient in need thereof.

25. (canceled)

26. The method according to claim 24, wherein:
(i) said cancer is selected from head and neck cancer, carcinoma of the bladder, breast, colon, kidney, epidermis, liver, lung, ovary, pancreas, stomach, thyroid, prostate, gastrointestinal system, or skin, a hematopoietic tumor of lymphoid or myeloid lineage, and a tumor of the central or peripheral nervous system; or
(ii) said cancer is selected from: colon adenocarcinoma, colon adenoma, colorectal carcinoma, small cell lung cancer, non-small cell lung carcinoma, exocrine pancreatic carcinoma, gastrointestinal stromal tumors, leukemia, acute lymphocytic leukemia, chronic lymphocytic leukemia, B-cell lymphoma, T-cell lymphoma, Burkett's lymphoma, acute myelogenous leukemia, chronic myelogenous leukemia, Imatinib sensitive and refractory chronic myelogenous leukemia, myeloproliferative disease, melanoma, bortezomib sensitive multiple myeloma, thyroid follicular cancer and glioma; or
(iii) said cancer is selected from: carcinoma of the prostate, gastrointestinal stromal tumors, acute lymphocytic leukemia, chronic lymphocytic leukemia, B-cell lymphoma, T-cell lymphoma, Burkett's lymphoma, acute myelogenous leukemia, chronic myelogenous leukemia, bortezomib sensitive multiple myeloma, non-small cell lung cancer, thyroid cancer, follicular cancer, melanoma, and ErbB2-positive breast cancer; or
(iv) said cancer is selected from: colon adenocarcinoma, colon adenoma, colorectal carcinoma, small cell lung cancer, non-small cell lung carcinoma, exocrine pancreatic carcinoma, gastrointestinal stromal tumors, leukemia, acute lymphocytic leukemia, chronic lymphocytic leukemia, B-cell lymphoma, T-cell lymphoma, Burkett's lymphoma, acute myelogenous leukemia, chronic myelogenous leukemia, Imatinib sensitive and refractory chronic myelogenous leukemia, myeloproliferative disease, melanoma, bortezomib sensitive multiple myeloma, thyroid follicular cancer and glioma; or
(v) said cancer is selected from refractory solid tumours, gastrointestinal stromal tumours (GIST), prostate cancer, melanoma (e.g. melanoma associated with BRAF mutation), non-small cell lung cancer (e.g. ALK-positive non-small cell lung cancer), HER2-positive breast cancer; and multiple myeloma; or
(vi) said cancer is selected from refractory solid tumours, gastrointestinal stromal tumours (GIST), prostate cancer, melanoma associated with BRAF mutation, and ALK-positive non-small cell lung cancer; or
(vii) said cancer is selected from refractory solid tumours, gastrointestinal stromal tumours (GIST), prostate cancer, melanoma (e.g. melanoma associated with BRAF mutation), non-small cell lung cancer (e.g. ALK-positive non-small cell lung cancer), HER2-positive breast cancer; and multiple myeloma.

27. (canceled)
